(19) United States
(12) Patent Application Publication
(10) Pub. No.: US 2009/0306238 A1
(43) Pub. Date: Dec. 10, 2009

(54) METHOD FOR THE PRODUCTION OF RIGID POLYURETHANE FOAMS

(75) Inventors: Stefan Dinsch, Schipkau (DE); Juergen Winkler, Schwarzheide (DE); Gerd Hoeppner, Schwarzheide (DE); Holger Seifert, Bohmte (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/513,446

(22) PCT Filed: Nov. 5, 2007
(86) PCT No.: PCT/EP2007/061862

§ 371 Date: May 4, 2009

(30) Foreign Application Priority Data
Nov. 14, 2006 (EP) 06124014.9

(51) Int. Cl. C08G 18/06; C07C 41/01

(52) U.S. Cl. 521/159; 568/679

(57) ABSTRACT

Process for producing rigid polyurethane foams by reacting
a) polyisocyanates with
b) compounds having at least two hydrogen atoms which are reactive toward isocyanate groups in the presence of
c) blowing agents,
   wherein the compounds having at least two hydrogen atoms which are reactive toward isocyanate groups comprise at least one polyether alcohol bi) which can be prepared by reacting aromatic amines with ethylene oxide and propylene oxide, with firstly propylene oxide and then ethylene oxide or a mixture of ethylene oxide and propylene oxide being added on in a first process step and the remaining amount of propylene oxide being added on in a second process step using a basic catalyst.

The invention relates to a process for producing rigid polyurethane foams by reacting polyisocyanates with compounds having at least two hydrogen atoms which are reactive toward isocyanate groups.

Rigid polyurethane foams have been known for a long time and are used primarily for insulation of heat and cold, e.g. in refrigeration appliances, in hot water storages, in district heating pipes or in building and construction, for example in sandwich elements. A summary overview of the production and use of rigid polyurethane foams may be found, for example, in Kunststoff-Handbuch, volume 7, Polyurethane 1st edition 1966, edited by Dr. R. Vieweg and Dr. A. Höchtlen, 2nd edition 1983, edited by Dr. Günter Oertel, and 3rd edition 1993, edited by Dr. Günter Oertel, Carl Hanser Verlag, Munich, Vienna.

In the use of rigid polyurethane foams, it is important to achieve optimal properties for the respective application.

In variation of the processing and use properties of rigid polyurethane foams, the compounds having at least two hydrogen atoms which are reactive toward isocyanate groups are most often adapted.

Particularly for use in refrigeration appliances, polyether alcohols obtained by addition of alkylene oxides onto aromatic amines are frequently used as compounds having at least two hydrogen atoms which are reactive toward isocyanate groups. Among the aromatic amines, the isomers of toluenediamine (TDA) are of particular importance. Such products result in optimal flow properties in the production of the foams and also a low thermal conductivity of the foams.

Polyether alcohols based on TDA and the preparation of such products are likewise known. Thus, EP 747 411 describes polyether alcohols based on ortho-TDA, also known as vicinal TDA. In the preparation of these polyether alcohols, pure ethylene oxide is added on in a first process step without use of a catalyst and pure propylene oxide is added on in a second process step using a basic catalyst.

The polyether alcohols prepared in this way have a relatively low functionality and result in late curing of the polyurethane system. It is therefore possible for even complicated hollow spaces as occur, in particular, in refrigeration appliances to be filled completely. The high content of ethylene oxide in the polyether alcohol leads to poor compatibility with the polyisocyanates and thus to poorer curing and a greater demolding thickness of the foams.

WO 05/044889, too, describes a rigid polyurethane foam which has been produced using a TDA-based polyether alcohol. Here, a mixture of ethylene oxide and propylene oxide is added on in a first reaction step and pure propylene oxide is added on in a second step. The proportion of ethylene oxide, based on the polyether alcohol, is from 2 to 25% by weight.

An important factor in many applications of rigid foams, especially in the insulation of refrigeration appliances, is the curing behavior of the system employed, which has a critical influence on the cycle times in appliance manufacture.

It is an object of the invention to develop a process for producing rigid polyurethane foams, which process results in good thermal conductivity and displays a high solubility of the blowing agents in the system and also optimal flow times and curing times. In addition, the polyols used should be compatible with one another.

This object has surprisingly been achieved by the use of a polyether alcohol which can be prepared by reacting aromatic amines with ethylene oxide and propylene oxide, with firstly propylene oxide and subsequently ethylene oxide being added on in a first process step and propylene oxide being added on in a second process step.

The invention accordingly provides a process for producing rigid polyurethane foams by reacting
a) polyisocyanates with
b) compounds having at least two hydrogen atoms which are reactive toward isocyanate groups in the presence of
c) blowing agents,
   wherein the compounds having at least two hydrogen atoms which are reactive toward isocyanate groups comprise at least one polyether alcohol bi) which can be prepared by reacting aromatic amines, preferably toluenediamine, with ethylene oxide and propylene oxide, with firstly propylene oxide and then ethylene oxide or a mixture of ethylene oxide and propylene oxide being added on in a first process step and the remaining amount of propylene oxide being added on in a second process step.

The invention further provides the polyether alcohols bi) and a process for preparing them by adding ethylene oxide and propylene oxide onto aromatic amines, wherein firstly propylene oxide and then ethylene oxide or a mixture of ethylene oxide and propylene oxide are/is added on in a first process step and the remaining amount of propylene oxide is added on in a second process step using a basic catalyst.

Reacting the amino groups of toluenediamine firstly with propylene oxide is particularly advantageously. This forms secondary hydroxyl groups. The further addition reaction of the subsequent ethylene oxide occurs preferentially at the remaining amino groups and binds primarily these. This results in a further minimization of free amino groups and thus to a further lowering of the intrinsic reactivity of the polyether alcohol. After propylene oxide has been fed in, pure ethylene oxide is, as described, preferably fed in. The introduction of mixtures of ethylene oxide and propylene oxide is also possible, in which case the content of propylene oxide in the mixture should not exceed 20% by weight, based on the mixture.

The hydroxyl number of the polyether alcohol bi) is preferably in the range from 140 to 480 mg KOH/g, in particular from 140 to 470 mg KOH/g. In a particularly preferred embodiment of the invention, the hydroxyl number can be, depending on the preferred field of use of the polyether alcohols prepared according to the invention, in the range from 140 to 180 mg KOH/g or in the range from 370 to 420 mg KOH/g.

As aromatic amines for preparing the polyether alcohols bi), it is possible to use diphenylmethane diamine (MDA) either alone or in a mixture with its higher homologues or toluenediamine (TDA). Preference is given to using TDA, with the TDA particularly preferably having a proportion of vicinal TDA of at least 95% by weight.

The first process step in the preparation of the polyether alcohols bi) is preferably carried out in the absence of a catalyst.

Furthermore, the second process step in the preparation of the polyether alcohols bi) is preferably carried out in the presence of a basic catalyst. As basic catalysts, preference is given to using alkaline compounds and/or amines, particularly preferably alkali metal and/or alkaline earth metal oxides and hydroxides and in particular potassium hydroxide.

Preference is given to using from 2.5 to 4 mol of alkylene oxide per mole of aromatic amine in the first process step. Particularly preference is given to using from 1.5 to 2 mol of ethylene oxide and from 1 to 2 mol of propylene oxide per mole of aromatic amine in the first process step.

The amount of ethylene oxide in the first phase is from 35 to 80% by weight, based on the weight of the total amount of alkylene oxide in the first phase. The ethylene oxide content of the polyether alcohol is preferably in the range from 4 to 20% by weight, based on the weight of the polyether alcohol.

In the second process step, a mixture of propylene oxide and from >0 to 20% by weight, preferably from >0 to 5% by weight, in each case based on the weight of the mixture, of ethylene oxide can be used in place of pure propylene oxide.

In a preferred embodiment of the preparation of the polyether alcohols bi), the second process step is carried out in the presence of water or at least one further compound having at least two active hydrogen atoms. The water can be added as such or as solvent for the alkaline catalyst.

The preparation of the polyether alcohols bi) is carried out by customary methods, for example as described in EP 747 411. Here, the addition reaction of the alkylene oxides is preferably carried out at a temperature in the range from 100 to 135° C. and a pressure in the range from 0.1 to 8 bar. The introduction of the alkylene oxides is usually followed by an after-reaction phase for the alkylene oxides to react completely. The crude polyether alcohol obtained in this way is freed of unreacted alkylene oxide and volatile compounds by distillation, preferably under reduced pressure, dewatered and worked up by acid neutralization and removal of the salts formed.

In the case of catalysis using tertiary amines, the crude product is stripped by means of nitrogen and/or under reduced pressure and subsequently filtered if appropriate.

The production of the rigid polyurethane foams is effected, as indicated above, by reacting the polyols with polyisocyanates, usually in the presence of catalysts, blowing agents and auxiliaries and/or additives.

The polyether alcohol bi) can here be used alone but is preferably used in the presence of further compounds having at least two hydrogen atoms which are reactive toward isocyanate groups.

As regards the other starting materials used for the process of the invention, the following details may be provided:

as organic polyisocyanates a), preference is given to using aromatic polyfunctional isocyanates.

Specific examples are: tolylene 2,4- and 2,6-diioscyanate (TDA) and the corresponding isomer mixtures, diphenylmethane 4,4′-, 2,4′- and 2,2′-diisocyanate (MDI) and the corresponding isomer mixtures, mixtures of diphenylmethane 4,4′- and 2,4′-diisocyanates, polyphenylpolymethylene polyisocyanates, mixtures of diphenylmethane 4,4′-, 2,4′- and 2,2′-diisocyanates and polyphenylpolymethylene polyisocyanates (crude MDI) and mixtures of crude (MDI) and tolylene diisocyanates. The organic diisocyanates and polyisocyanates can be used individually or in the form of mixtures.

Use is frequently also made of modified polyfunctional isocyanates, i.e. products obtained by chemical reaction of organic diisocyanates and/or polyisocyanates. Examples which may be mentioned are diisocyanates and/or polyisocyanates comprising isocyanurate and/or urethane groups. The modified polyisocyanates may, if appropriate, be mixed with one another or with unmodified organic polyisocyanates such as diphenylmethane 2,4′-, 4,4′-diisocyanate, crude MDI, tolylene 2,4- and/or 2,6-diisocyanate.

It is also possible to use reaction products of polyfunctional isocyanates with polyfunctional polyols, or mixtures thereof with other diisocyanates and polyisocyanates.

Crude MDI having an NCO content of from 29 to 33% by weight and a viscosity at 25° C. in the range from 150 to 1000 mPa·s has been found to be particularly useful as organic polyisocyanate.

As compounds having at least two hydrogen atoms which are reactive toward isocyanate b) which can be used together with the polyether alcohols bi) used according to the invention, use is made of, in particular, polyether alcohols and/or polyester alcohols having OH numbers in the range from 100 to 1200 mg KOH/g.

The polyester alcohols used together with the polyether alcohols bi) used according to the invention are usually prepared by condensation of polyfunctional alcohols, preferably diols, having from 2 to 12 carbon atoms, preferably from 2 to 6 carbon atoms, with polyfunctional carboxylic acids having from 2 to 12 carbon atoms, for example succinic acid, glutaric acid, adipic acid, suberic acid, azelaic acid, sebacic acid, decanedicarboxylic acid, maleic acid, fumaric acid and preferably phthalic acid, isophthalic acid, terephathalic acid and the isomeric naphthalenedicarboxylic acids.

The polyether alcohols used together with the polyether alcohols bi) used according to the invention usually have a functionality of from 2 to 8, in particular from 3 to 8.

In particular, use is made of polyether alcohols prepared by known methods, for example by anionic polymerization of alkylene oxides in the presence of catalysts, preferably alkaline metal hydroxides.

Alkylene oxides used are usually ethylene oxide and/or propylene oxide, preferably pure 1,2-propylene oxide.

As starter molecules, use is made of, in particular, compounds having at least 3, preferably from 4 to 8, hydroxyl groups or at least two primary amino groups in the molecule.

As starter molecules having at least 3, preferably from 4 to 8, hydroxyl groups in the molecule, preference is given to using trimethylopropane, glycerol, pentaerythritol, sugar compounds such as glucose, sorbitol, mannitol and sucrose, polyhydric phenols, resoles such as oligomeric condensation products of phenol and formaldehyde and Mannich condensates of phenols, formaldehyde and dialkanolamines and also melamine.

As starter molecules having at least two primary amino groups in the molecule, preference is given to using aromatic diamines and/or polyamines, for example phenylenediamines, 2,3-, 2,4-, 3,4- and 2,6-toluenediamine and 4,4′-, 2,4′- and 2,2′-diaminodiphenylmethane, and aliphatic diamines and polyamines such as ethylenediamine.

The polyether alcohols have a functionality of preferably from 3 to 8 and hydroxyl numbers of preferably from 100 mg KOH/g to 1200 mg KOH/g and in particular from 120 mg KOH/g to 570 mg KOH/g.

The use of bifunctional polyols, for example polyethylene glycols and/or polypropylene glycols, having a molecular weight in the range from 500 to 1500 in the polyol component enables the viscosity of the polyol component to be adapted.

The compounds having at least two hydrogen atoms which are reactive toward isocyanate b) also include the chain extenders and crosslinkers which may be concomitantly used. The rigid PUR foams can be produced without or with concomitant use of chain extenders and/or crosslinkers. The addition of bifunctional chain extenders, trifunctional and higher-functionality crosslinkers or, if appropriate, mixtures thereof can prove to be advantageously for modifying the mechanical properties. As chain extenders and/or crosslinkers, preference is given to using alkanolamines and in particular diols and/or triols having molecular weights of less than 400, preferably from 60 to 300.

Chain extenders, crosslinkers or mixtures thereof are advantageously used in an amount of from 1 to 20% by weight, preferably from 2 to 5% by weight, based on the polyol component b).

Further information regarding the polyether alcohols and polyester alcohols used and their preparation may be found, for example, in Kunststoffhandbuch, volume 7 “Polyurethane”, edited by Günter Oertel, Carl-Hanser-Verlag, Munich, 3rd edition 1993.

In a particularly preferred embodiment of the process of the invention, the component b) comprises a polyether alcohol bii) which can be prepared by reacting H-functional sugars with alkylene oxides.

The sugars used for preparing the polyether alcohol bii) are preferably sucrose and/or sorbitol. The polyether alcohols bii) preferably have a hydroxyl number in the range from 350 to 800 mg KOH/g.

Catalysts used are, in particular, compounds which strongly accelerate the reaction of the isocyanate groups with the groups which are reactive toward isocyanate groups. Such catalysts are strongly basic amines, e.g. secondary aliphatic amines, imidazoles, amidines and alkanolamines, or organic metal compounds, in particular organic tin compounds.

If isocyanurate groups are also to be incorporated into the rigid polyurethane foam, specific catalysts are required for this purpose. As isocyanurate catalysts, use is usually made of metal carboxylates, in particular potassium acetate and its solutions.

The catalysts can, depending on requirements, be used either alone or in any mixtures with one another.

As blowing agent c), preference is given to using water which reacts with isocyanate groups to eliminate carbon dioxide. It is also possible to use physical blowing agents in combination with or instead of water. These are compounds which are inert toward the starting components and are usually liquid at room temperature and vaporize under the conditions of the urethane reaction. The boiling point of these compounds is preferably below 50° C. Physical blowing agents also include compounds which are gaseous at room temperature and are introduced under pressure into the starting components or are dissolved therein, for example carbon dioxide, low-boiling alkanes and fluoroalkanes.

The physical blowing agents are usually selected from the group consisting of alkanes and cycloalkanes having at least 4 carbon atoms, dialkyl ethers, esters, ketones, acetals, fluoroalkanes having from 1 to 8 carbon atoms and tetraalkylsilanes having from 1 to 3 carbon atoms in the alkyl chain, in particular tetramethylsilane.

Examples which may be mentioned are propane, n-butane, isobutane and cyclobutane, n-pentane, isopentane and cyclopentane, cyclohexane, dimethyl ether, methyl ethyl ether, methyl butyl ether, methyl formate, acetone and also fluoroalkanes which are degraded in the troposphere and therefore do not harm the ozone layer, e.g. trifluoromethane, difluormethane, 1,1,1,3,3-pentafluorobutane, 1,1,1,3,3-pentafluoropropane, 1,1,1,2-tetrafluoroethane, difluoroethane and 1,1,1,2,3,3,3-heptafluoropropane and also perfluoroalkanes such as C₃F₈, C₄F₁₀, C₅F₁₂, C₆F₁₄, and C₇F₁₇. Particular preference is given to pentanes, in particular cyclopentane. The physical blowing agents mentioned can be used either alone or in any combinations with one another.

The process of the invention can, if required, be carried out in the presence of flame retardants and also customary auxiliaries and/or additives.

As flame retardants, it is possible to use organic phosphoric and/or phosphonic esters. Preference is given to using compounds which are not reactive toward isocyanate groups. Preferred compounds also include chlorine-comprising phosphoric esters.

Typical representatives of this group of flame retardants are triethyl phosphate, diphenyl cresyl phosphate, tris(chloropropyl) phosphate and diethyl ethanephosphonate.

It is also possible to use bromine-comprising flame retardants. As bromine-comprising flame retardants, preference is given to using compounds which have groups which are reactive toward the isocyanate group. Such compounds are esters of tetrabromophthalic acid with aliphatic diols and alkoxylation products of dibromobutenediols. Compounds derived from the series of brominated, OH-comprising neopentyl compounds can also be employed.

Auxiliaries and/or additives used are the materials known per se for this purpose, for example surface-active substances, foam stabilizers, cell regulators, fillers, pigments, dyes, hydrolysis inhibitors, antistatics, fungistatic and bacteriostatic substances.

Further details regarding the starting materials, blowing agents, catalysts and auxiliaries and/or additives used for carrying out the process of the invention may be found, for example, in Kunststoffhandbuch, volume 7, “Polyurethane” Carl-Hanser-Verlag, Munich, 1st edition, 1966, 2nd edition, 1983, and 3rd edition, 1993.

To produce the rigid polyurethane foams, the polyisocyanates a) and the compounds having at least two hydrogen atoms which are reactive toward isocyanate groups b) are reacted in such amounts that the isocyanate index is in the range from 100 to 220, preferably from 115 to 195. The rigid polyurethane foams can be produced batchwise or continuously with the aid of known mixing apparatuses.

The production of polyisocyanurate foams can also be carried out at a higher index, preferably up to 350.

The rigid PUR foams according to the invention are usually produced by the two-component process. In this process, the compounds having at least two hydrogen atoms which are reactive toward isocyanate groups b) are mixed with the flame retardants, the catalysts, the blowing agents and the further auxiliaries and/or additives to form a polyol component and this is reacted with the polyisocyanates or mixtures of the polyisocyanates and, if appropriate, blowing agents, also referred to as isocyanate component.

The starting components are usually mixed at a temperature of from 15 to 35° C., preferably from 20 to 30° C. The reaction mixture can be introduced into closed support tools by means of high- or low-pressure metering machines. For example, sandwich elements are manufactured discontinuously by this technology.

The polyether alcohols prepared by the process of the invention can, as described, be reacted with polyisocyanates to form rigid polyurethane foams. They are very readily miscible with the other constituents of the polyol component and readily compatible with the hydrocarbons which are preferably used as blowing agents for producing rigid PU foams. The foams have a low thermal conductivity, good flowability, good curing and good demoldability.

The invention is illustrated by the following examples.

### EXAMPLE 1

### According to the Invention

43.6 kg of vicinal toluenediamine were placed in a 250 l pressure autoclave provided with a tiered stirrer (250 rpm), reactor heating and cooling, metering facilities for solid and liquid substances and alkylene oxides and also facilities for blanketing with nitrogen and a vacuum system and were heated to 90° C. 22.0 kg of propylene oxide were subsequently metered in and reacted at 130° C. 10 minutes after the end of the metered addition, the reactor was filled with 2.5 bar of nitrogen and 28.7 kg of ethylene oxide were metered in at 130° C. After an after-reaction time of 3 hours, 0.705 kg of 48% strength potassium hydroxide solution was added to the reaction mixture and mixed in. 100.2 kg of propylene oxide was subsequently metered in while ensuring that the pressure did not exceed 6.5 bar. During the reaction, the reaction temperature rose to 135° C. and was maintained for the entire reaction time. An after-reaction time of 3 hours at 135° C. followed.

The crude polyetherol was hydrolyzed by means of water, neutralized with phosphoric acid, filtered and vacuum-stripped.

The end product had the following values:

| | | |
|---|---|---|
| Hydroxyl number: | 391 | mg KOH/g |
| Amine number: | 105 | mg KOH/g |
| Water content: | 0.044% | by weight |
| Viscosity at 25° C.: | 11 144 | mPa s |

### EXAMPLE 2

### According to the Invention

43.6 kg of viscinal toluenediamine were placed in a 250 l pressure autoclave provided with a tiered stirrer (250 rpm), reactor heating and cooling, metering facilities for solid and liquid substances and alkylene oxides and also facilities for blanketing with nitrogen and a vacuum system and were heated to 90° C. 22.0 kg of propylene oxide were subsequently metered in and reacted at 110° C. 10 minutes after the end of the metered addition, the reactor was filled with 2.5 bar of nitrogen and 28.7 kg of ethylene oxide were metered in at 110° C. After an after-reaction time of 3 hours, 1.945 kg of dimethylethanolamine were added to the reaction mixture and mixed in. 100.2 kg of propylene oxide was subsequently metered in while ensuring that the pressure did not exceed 6.5 bar. During the reaction, the reaction temperature rose to 112° C. and was maintained for the entire reaction time. An after-reaction time of 3 hours at 120° C. followed.

The crude polyetherol obtained was vacuum-stripped and filtered.

The end product had the following values:

| | | |
|---|---|---|
| Hydroxyl number: | 395 | mg KOH/g |
| Amine number: | 111 | mg KOH/g |
| Water content: | 0.032% | by weight |
| Viscosity at 25° C.: | 25 620 | mPa s |

### EXAMPLE 3

### According to the Invention

17.3 kg of viscinal toluenediamine were placed in a 2501 pressure autoclave provided with a tiered stirrer (250 rpm), reactor heating and cooling, metering facilities for solid and liquid substances and alkylene oxides and also facilities for blanketing with nitrogen and a vacuum system and were heated to 90° C. 8.75 kg of propylene oxide were subsequently metered in and reacted at 130° C. 10 minutes after the end of the metered addition, the reactor was filled with 2.5 bar of nitrogen and 11.4 kg of ethylene oxide were metered in at 130° C. After an after-reaction time of 3 hours, 0.80 kg of 48% strength potassium hydroxide solution was added to the reaction mixture and mixed in. 160 kg of propylene oxide was subsequently metered in while ensuring that the pressure did not exceed 6.5 bar. During the reaction, the reaction temperature rose to 135° C. and was maintained for the entire reaction time. An after-reaction time of 3 hours at 135° C. followed.

The crude polyetherol was hydrolyzed by means of water, neutralized with phosphoric acid, filtered and vacuum-stripped.

The end product had the following values:

| | | |
|---|---|---|
| Hydroxyl number: | 161 | mg KOH/g |
| ph value: | 8.5 | |
| Water content: | 0.07% | by weight |
| Viscosity at 25° C.: | 834 | mPa s |

### EXAMPLE 4

### Production of a Rigid Polyurethane Foam

The following components were mixed to form a polyol component:

58.75 parts of sucrose/glycerol-initiated PO polyetherol having an OH number of 450 mg KOH/g
10.00 parts of polyether alcohols from example 2
25.00 parts of TDA-initiated PO/EO/PO polyetherol having an OH number of

### 160 mg KOH/g

2.50 parts of Tegostab B8462
0.50 part of PMDETA
0.50 part of DMCHA
0.40 part of 1,3,5,tris-(3-dimethylaminopropyl)hexahydro-s-triazine
2.35 parts of water

This mixture was foamed with cyclopentane/isopentane and Lupranat® M20s in a weight ratio of 100:17:143 on a Puromat® 80/30 high-pressure metering unit.

The following values were determined:

| | |
|---|---|
| Cream time: | 7 s |
| Fiber time: | 39 s |

Demolding thickness (90 mm mold) at 15% overfilling:

| | |
|---|---|
| 4 min: | 93.1 mm |
| 5 min: | 92.0 mm |
| Thermal conductivity: | 19.2 mW/mK at a mean temperature of 23° C. |
| Compressive strength: | 0.13 N/mm² |

1. A process for producing rigid polyurethane foams by reacting
a) polyisocyanates with
b) compounds having at least two hydrogen atoms which are reactive toward isocyanate groups in the presence of
c) blowing agents,
wherein the compounds having at least two hydrogen atoms which are reactive toward isocyanate groups comprise at least one polyether alcohol bi) which can be prepared by reacting aromatic amines with ethylene oxide and propylene oxide, with firstly propylene oxide and then ethylene oxide or a mixture of ethylene oxide and propylene oxide being added on in a first process step and the remaining amount of propylene oxide being added on in a second process step.

2. The process according to claim 1, wherein the first process step in the preparation of the polyether alcohols bi) is carried out in the absence of a catalyst.

3. The process according to claim 1, wherein the second process step in the preparation of the polyether alcohols bi) is carried out in the presence of a basic catalyst.

4. The process according to claim 1, wherein firstly propylene oxide and subsequently ethylene oxide are fed in the first process step at a proportion of ethylene oxide of from 35 to 80% by weight, based on the amount of alkylene oxide in the first process step.

5. The process according to claim 1, wherein the content of ethylene oxide in the mixture in the second process step is from >0 to 5% by weight, based on the weight of the mixture of ethylene oxide and propylene oxide in the second process step.

6. The process according to claim 1, wherein from 2.5 to 4 mol of alkylene oxide are used per mole of aromatic amine in the first process step.

7. The process according to claim 1, wherein from 1.5 to 2 mol of ethylene oxide and from 1 to 2 mol of propylene oxide are used per mole of aromatic amine in the first process step.

8. The process according to claim 1, wherein the second process step is carried out in the presence of water or at least one further compound having at least two active hydrogen atoms.

9. The process according to claim 1, wherein the total content of ethylene oxide in the polyether alcohol is from 4 to 20% by weight, based on the weight of the polyether alcohol.

10. The process according to claim 1, wherein the hydroxyl number of the polyether alcohol bi) is in the range from 140 to 480 mg KOH/g.

11. The process according to claim 1, wherein the hydroxyl number of the polyether alcohol bi) is in the range from 140 to 420 mg KOH/g.

12. The process according to claim 1, wherein toluenediamine is used as aromatic amine for preparing the polyether alcohol bi).

13. The process according to claim 9, wherein toluenediamine having a proportion of vicinal toluenediamine of at least 95% by weight is used as aromatic amine for preparing the polyether alcohol bi).

14. The process according to claim 1, wherein the component b) comprises a polyether alcohol bii) which can be prepared by reacting H-functional sugars with alkylene oxides.

15. The process according to claim 13, wherein the sugars used for preparing the polyether alcohol bii) are sucrose and/or sorbitol.

16. A process for preparing polyether alcohols by adding ethylene oxide and propylene oxide onto aromatic amines, wherein firstly propylene oxide and then ethylene oxide or a mixture of ethylene oxide and propylene oxide are/is added on in a first process step and the remaining amount of propylene oxide is added on in a second process step using a basic catalyst.

17. A polyether alcohol which can be prepared according to claim 16.
